(19) United States
(12) Patent Application Publication
(10) Pub. No.: US 2004/0068186 A1
(43) Pub. Date: Apr. 8, 2004

(54) Ultrasonic therapeutic probe and ultrasonic device

(76) Inventors: Kazunari Ishida, Chiba (JP); Yutaka Sato, Chiba (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/466,199

(22) PCT Filed: Jan. 22, 2002
(86) PCT No.: PCT/JP02/00422

(30) Foreign Application Priority Data
Jan. 22, 2001 (JP) 2001-13650

(51) Int. Cl. A61B 8/00; A61N 7/00

(52) U.S. Cl. 600/439; 601/2

(57) ABSTRACT

According to the present invention, a therapeutic probe 1 includes a diagnostic probe 2, a therapeutic transducer 3, and a supporting member for supporting them, wherein the therapeutic transducer has a plurality of transducer elements, and a focal point at which ultrasonic waves transmitted by the respective transducer elements converge can be freely shifted by controlling the timing of supplying driving signals to the respective transducer elements. By locating the focal point of ultrasonic beams of the therapeutic transducer 3 on the plane scanned by ultrasonic beams of the diagnostic probe 2, the ultrasonic therapy can be performed while substantially observing the portion to be treated through the diagnostic image.

### TECHNICAL FIELD

The present invention relates to an ultrasonic therapeutic probe and an ultrasonic therapeutic apparatus suitable for performing treatment by transmitting high-energy ultrasonic waves to a lesion within a body.

### BACKGROUND OF THE INVENTION

As a method of treating a lesion within a living body, there has been proposed a therapy in which high-energy ultrasonic waves are transmitted from outside the body to the lesion so as to heat and solidify the lesion or to cauterize it. This method of ultrasonic therapy is performed using an ultrasonic therapeutic probe (hereinafter referred to as a therapeutic probe) that integrally incorporates a diagnostic probe for imaging an ultrasonic diagnostic image and a therapeutic transducer for transmitting the high-energy ultrasonic waves to a lesion identified from the above-obtained diagnostic image.

In the proposed therapeutic transducer, an ultrasonic transmission plane is conventionally formed on a curved plane having a curvature radius R so that ultrasonic beams transmitted from the transmission plane converge at the plane's curvature center (focal point), and that focal point is made to coincide with the portion to be treated, so that the ultrasonic energy to be irradiated to the treated portion is thus increased.

However, when the therapeutic transducer with a curved plane is formed by one sheet of a plane-shaped transducer, the portion to be treated is limited to a position determined by the transducer curvature R, which is only one point. Therefore, it is necessary to prepare plural kinds of therapeutic probes or therapeutic transducers having different focal distances, and to change the therapeutic probe or the diagnostic transducer in accordance with the depth of the portion diagnosed with the diagnostic probe. Consequently, it takes time to complete the treatment, and the patient may feel pain.

Further, since the therapeutic probe generally is portable for the sake of greater usability, therapeutic ultrasonic waves might be transmitted to a portion other than the portion which must be treated, due to hand movement, if the time phase (time point) of diagnosis is different from that of treatment.

The object of the present invention is to enable transmission of therapeutic ultrasonic waves with one therapeutic probe to portions to be treated which are at different depths.

Another object thereof is to enable ultrasonic therapy while substantially observing the diagnosed portion through a diagnostic image.

### SUMMARY OF THE INVENTION

A therapeutic probe according to the present invention includes a diagnostic probe; a therapeutic transducer; and a supporting member for supporting the diagnostic probe and the therapeutic transducer, wherein the therapeutic transducer is formed of a plurality of transducer elements separated from each other, and the respective transducer elements are connected to distribution lines to which a driving signal is supplied.

According to thus-constructed therapeutic probe, by adjusting the phase of driving signals to be supplied to the respective transducer elements the focal point on which the ultrasonic waves transmitted from the respective transducer elements converge can be freely shifted. Therefore, one therapeutic probe can transmit the therapeutic ultrasonic waves to the portions to be treated at various depths.

Further, an ultrasonic therapeutic apparatus according to the invention includes a transmitting circuit for outputting an ultrasound-driving signal to the diagnostic probe; a receiving circuit for receiving and processing a received signal that has been output from the diagnostic probe; an image processing unit for generating a diagnostic image in accordance with the received signal that is processed by the receiving circuit; a display unit for displaying the diagnostic image generated by the image processing unit; a therapeutic wave transmitting circuit for outputting an ultrasound-driving signal to be supplied to respective transducer elements of the therapeutic transducer on which a plurality of transducer elements are arranged; and a control unit for controlling the transmitting circuit, the receiving circuit, the image processing unit, and the therapeutic wave transmitting circuit, wherein the control unit has means for adjusting the phase of the driving signal to be supplied to each of the transducer elements by controlling the therapeutic transmitting circuit and controlling the focal point the ultrasonic beams transmitted by the respective transducer elements.

In the above-described case, the therapeutic transducer may formed such that the surface from which ultrasound waves are emitted is a plain surface or a concave surface. Further, the therapeutic transducer preferably has a width direction and a longitudinal direction, and is divided in the longitudinal direction into plural parts. In such a case, the transmitting plane preferably has a concave curvature in the width direction. Further, the therapeutic transducer and the diagnostic probe preferably are integrally constructed. Particularly, they preferably are constructed integrally such that the focus of the ultrasonic beams transmitted by the therapeutic transducer are located on the plane scanned by the ultrasonic beams transmitted by the diagnostic probe.

### BRIEF DESCRIPTION OF THE DRAWINGS

FIG. 1 is a cross sectional view of a schematic diagram showing the structure of an ultrasonic therapeutic probe according to one embodiment of the present invention;

FIG. 2 is an explanatory diagram of focus adjustment of the ultrasonic therapeutic probe shown in FIG. 1;

FIG. 3 is a diagram showing the structure of an ultrasonic therapeutic apparatus according to one embodiment of the invention;

FIG. 4 is a time chart showing the operation in the embodiment show in FIG. 3;

FIG. 5 is a schematic diagram showing the structure of an ultrasonic therapeutic probe according to one embodiment of the invention; and

FIG. 6 is a schematic diagram showing ultrasonic therapy according to the embodiment of the invention.

### BEST MODE FOR CARRYING OUT THE INVENTION

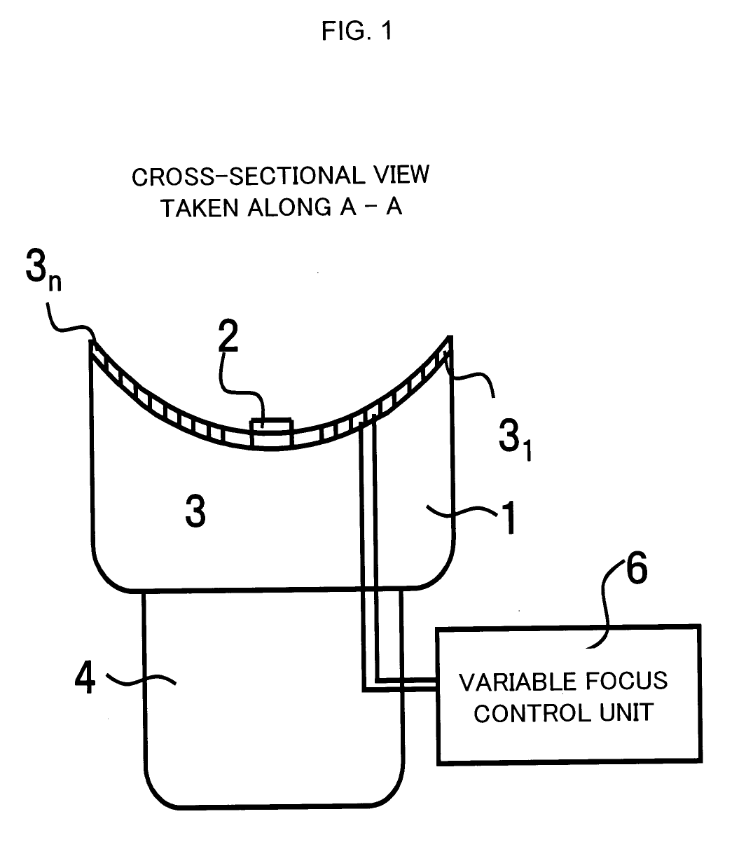

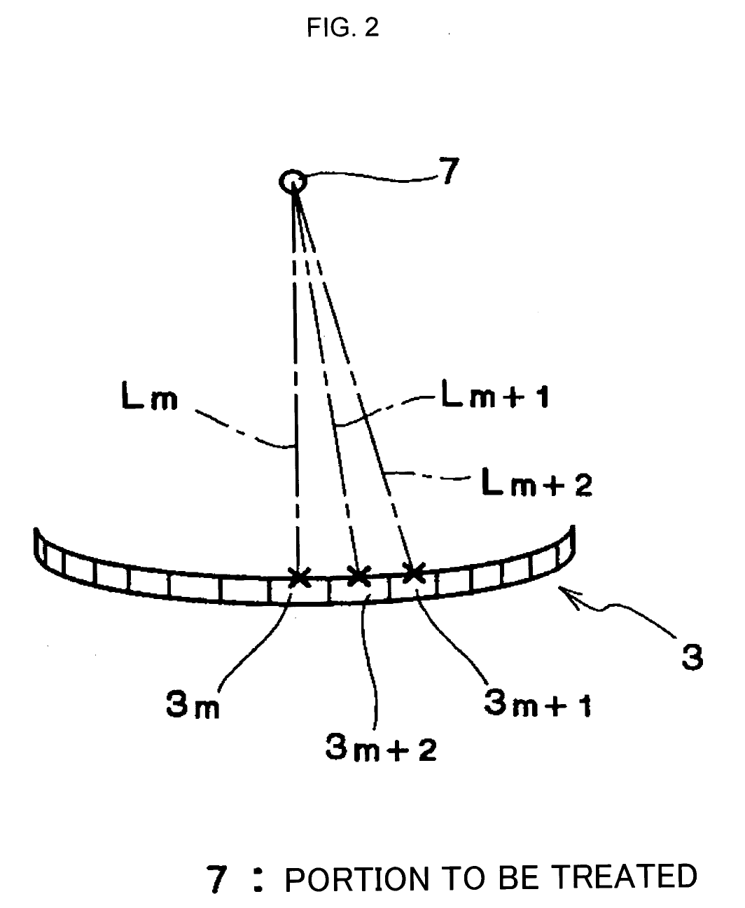

Hereinafter, the present invention will be described in accordance with an embodiment. FIG. 1 shows the structure of an ultrasonic therapeutic probe according to the invention, and FIG. 2 shows the adjusting operation of the portion to be treated using the ultrasonic therapeutic probe.

### Structure of the Apparatus

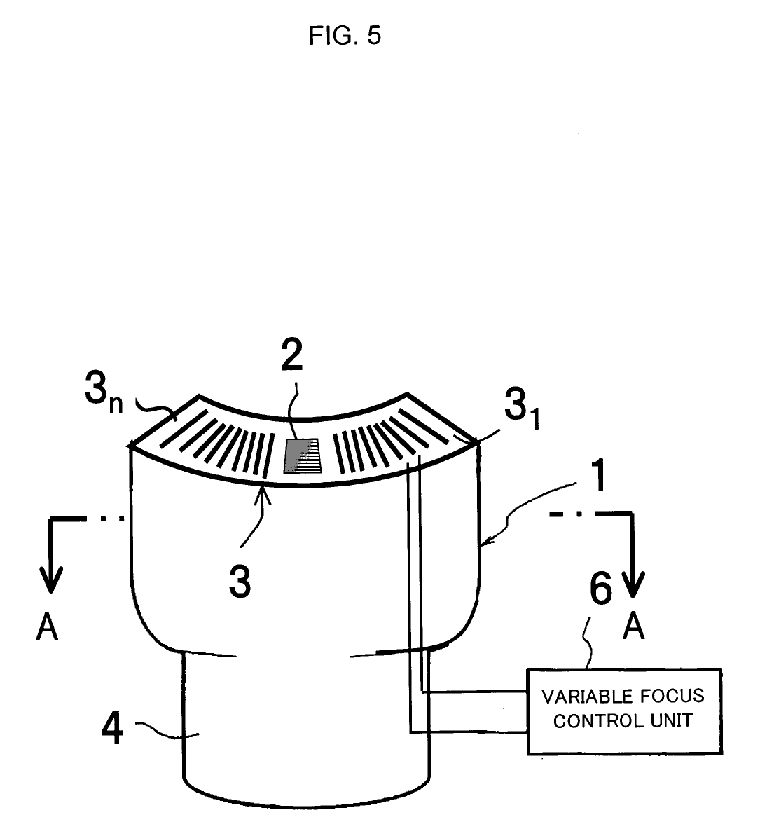

As shown in FIG. 1 and FIG. 5, a therapeutic probe 1 includes a diagnostic probe 2, a therapeutic transducer 3, a probe supporter 4, a probe cover, and a variable focus control unit 6. In the same manner as a diagnostic probe used in a known ultrasonic diagnostic apparatus, for example, the diagnostic probe 2 is formed of a plurality of transducers that is arranged in a line in a convex shape and installed on the probe supporter 4. On the therapeutic transducer 3, a plurality of transducer elements 3ᵢ, . . . , 3ₙ are divided into both sides, symmetrically arranged with respect to the center and installed on the probe supporter 4. Consequently, the focus of the ultrasonic beams transmitted by the therapeutic transducers is always on the center of the diagnostic image. Therefore, the diagnostic probe 2 and the therapeutic transducer 3 are integrally constructed on the probe supporter 4. Further, an ultrasound-transmitting plane of the plurality of transducer elements 3₁, . . . , 3ₙ forms a concave surface. Incidentally, in the drawing, the arranging direction of the plural transducer elements of the therapeutic transducer 3 is perpendicular to that of the transducers of the diagnostic probe 2. However, the invention is not limited thereto.

At the front of thus constructed diagnostic probe 2 and therapeutic transducer 3 is provided a probe cover made of a material that can easily match the acoustic impedance of the living body. Inside of the probe cover is filled with medium such as degasified water so as to easily transmit ultrasound. The probe supporter 4 is shaped such that it can be held by hand. Consequently, treatment can be performed with the therapeutic probe 1 held by the hand, whereby flexibility of the treatment is improved.

The variable focus control unit 6 is designed to supply an ultrasound-driving pulse for driving the therapeutic transducer 3 to the respective transducer elements 3₁, . . . , 3ₙ. Particularly, the variable focus control unit 6 adjusts the phase of the driving pulse to be supplied to the respective transducer elements 3₁, . . . , 3ₙ, and thus controls the focal point of the beams formed by ultrasonic waves transmitted by the transducer elements 3₁, . . . , 3ₙ to be on the portion 7 to be treated.

Here, the operation principle of the variable focus control unit 6 that variably controls the focal point of the ultrasonic beams will be described with reference to FIG. 2. FIG. 2 schematically shows the therapeutic transducer 3. Each of the transducer elements 3₁, . . . , 3ₙ has its own size, and each ultrasonic wave transmitted from one transducer element can be approximated as one transmitted from the respective sound source point shown with marks X in the drawing. It is given that the transducer element 3ₘ on the central portion of the therapeutic transducer 3 is a center of a coordinate system, and the coordinates thereof are (0, 0). The portion 7 to be treated is located on a coordinate (0, Lₘ), the distance Lₘ away from the transducer element 3ₘ in the vertical direction. When the sound source point coordinate of an arbitrary transducer element 3ₘ₊₁ is (x₁, y₁), the distance Lₘ₊₁ from this point to the portion 8 to be treated is represented by the next formula (1):

Lₘ₊₁=√(x₁²+(Lₘ−y₁)²) (1)

Here, given that the sound velocity in the ultrasound propagation medium is represented by C, the ultrasonic propagation time Tm from the transducer element 3ₘ to the portion 7 to be treated is represented as below:

Tₘ=Lₘ/C

And the ultrasonic propagation time Tₘ₊₁ from an arbitrary transducer element 3ₘ₊₁ to the portion 8 to be treated is represented as below:

Tₘ₊₁=Lₘ₊₁/C

Here, when Tₘ₊₁>Tₘ, propagation time from the transducer element 3ₘ₊₁ is greater than from the transducer element 3ₘ. Then, by transmitting an ultrasonic wave from the transducer element 3ₘ₊₁ ahead of the transducer element 3ₘ by the time difference τₘ₊₁=Tₘ₊₁−Tₘ, the ultrasonic waves arrive at the portion 7 to be treated at the same time. A similar calculation is done for every transducer element, and the timing of ultrasonic transmission from every transducer element is controlled such that every ultrasound can arrive at the portion 7 to be treated at the same time. In this manner, the ultrasonic waves from the transducer elements are converged at the portion 7 to be treated, and strong ultrasonic energy is given to that portion. When the location of the portion 7 to be treated is shifted and Lₘ is thus shifted, the ultrasonic transmission timing, that is, the timing of ultrasonic pulse application for driving the transducer elements is controlled according to the above calculation.

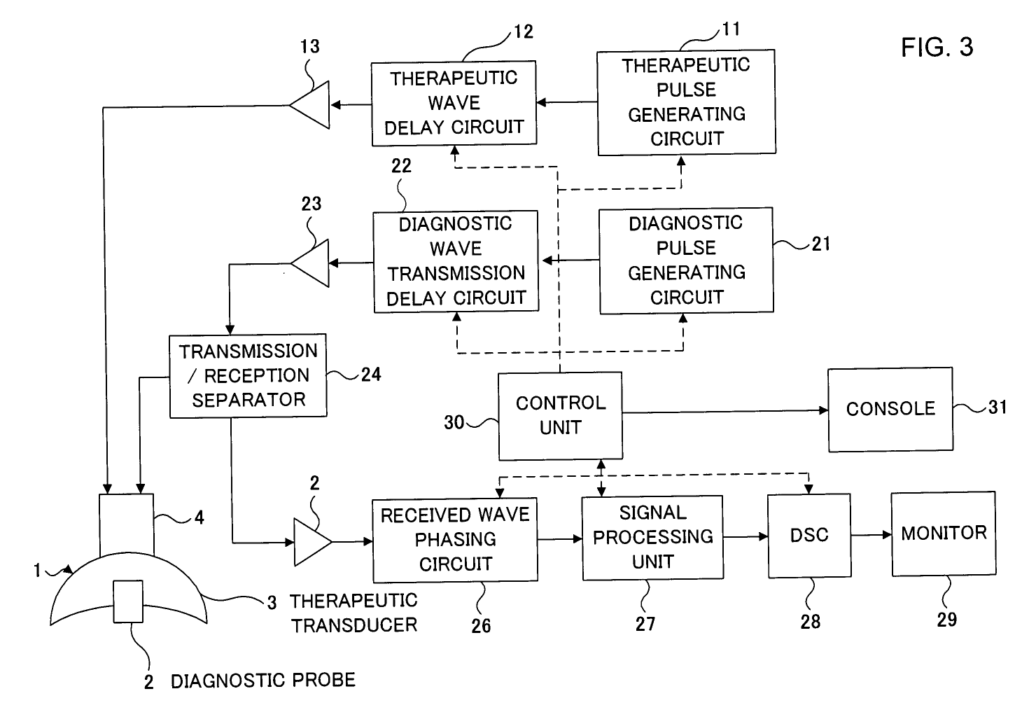

Next, FIG. 3 shows an embodiment of the ultrasonic therapeutic apparatus to which the therapeutic probe according to the above embodiment is applied. In FIG. 2, components having the same function or structure as in the embodiment in FIG. 1 are provided with the same reference numbers, and description thereof will be omitted. The therapeutic transducer 3 on the therapeutic probe 1 is designed to be supplied with ultrasonic pulses generated by a therapeutic pulse generating circuit 11 through a therapeutic wave delay circuit 12 and an amplifier 13. That is, the ultrasonic waves are delay-controlled by the therapeutic delay circuit 12 for the respective transducer elements, converted into driving pulses with high energy by the amplifier 13, and supplied to the respective transducer elements. Incidentally, the therapeutic wave delay circuit 12 and the amplifier 13 basically correspond to the variable focus control unit 6 shown in FIG. 1.

On the other hand, a diagnostic ultrasonic pulse generated by a diagnostic pulse generating circuit 21 is focus-processed by a diagnostic wave transmission delay circuit 22, amplified by an amplifier 23, and supplied to transducer elements that form the diagnostic probe 2 through a transmission/reception separator 24. The signals received from the living body by the diagnostic probe 2 are led to an amplifier 25 through the transmission/reception separator 24 and amplified thereby, and converted into a signal that emphasizes the signals received from a desired portion within the living body by adjusting the phase of the received signals at a received wave phasing circuit 26. In accordance with the received signal output by the received wave phasing circuit 26, a diagnostic image is generated by a signal processing unit 27 and a DSC (digital scan converter) 28, and it is displayed on a monitor 29.

The above therapeutic pulse generating circuit 11, the therapeutic wave delay circuit 12, the diagnostic pulse generating circuit 21, the diagnostic wave transmission delay circuit 22, the received wave phasing circuit 26, the signal processing unit 27, and the DSC 28 are controlled by commands of a control unit 30 including a computer. An operator can set various kinds of diagnostic conditions and therapeutic conditions by inputting commands from a console 31 to the control unit 30.

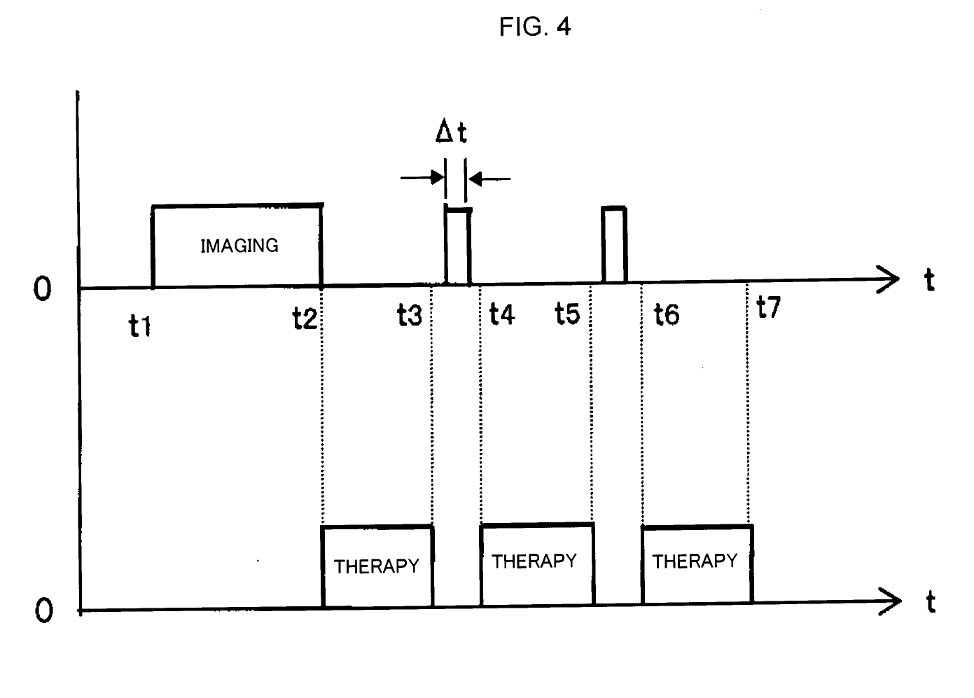

Next, the operation involved in performing ultrasonic therapy by using the thus-constructed ultrasonic therapeutic apparatus will be described with reference to a time chart in FIG. 4. In FIG. 4, the horizontal axis represents time and the vertical axis shows which operation is being carried out. First, the therapeutic probe 1 is attached to the body surface of an object to be examined, or to the surface of an organ if the body's viscera is opened in an operation, and is held toward the area of the body including the portion to be treated.

### Observation of the Portion to be Treated: t1 to t2

First, when a command to begin imaging is input from the console 31 so as to image the portion to be treated in advance of treatment, the control unit 30 outputs a command to the diagnostic pulse generating circuit 21 and the diagnostic wave transmission delay circuit 23 in response to the command. In this manner, the diagnostic pulse generating circuit 21 and the diagnostic transmission wave delay circuit 23 operate, and ultrasonic beams are transmitted by the diagnostic probe 2 to the interior of the body to be examined. The ultrasonic beams perform scanning in the arranging direction of transducers of the diagnostic probe 2, and the ultrasonic beam is transmitted to a region along a sectoral cross-sectional plane of the object. Ultrasonic waves reflecting from the region where the ultrasonic waves have been transmitted are received by the transducers of the diagnostic probe 2. These received signals for the respective ultrasonic beams are phased by the received wave phasing circuit 26. A two-dimensional image of the cross-sectional plane is generated by the image processing unit formed of the signal processing unit 27 and the DSC 28, and it is displayed on the monitor 29. In this manner, the interior of the living body is diagnosed by observing the cross-sectional image. When a portion to be treated appears on the cross-sectional image, treatment is performed.

### Treatment Operation t2 to t3

When a portion to be treated appears on the cross-sectional image, the therapeutic probe 1 is held on the present position. First, the control unit 30 calculates, for example, the distance Lₘ from the transducer element 3ₘ at the center of the therapeutic transducer 3 to the portion to be treated 7. Then, the delay times τ₁ to τₙ of the driving pulses to be supplied to the respective transducer elements 3₁ to 3ₙ, delayed with respect to the driving pulse supplied to the therapeutic transducer element 3ₘ, are calculated and output to the therapeutic wave delay circuit 12. The therapeutic wave delay circuit 12 sequentially outputs the driving pulses to be supplied to the respective therapeutic transducer elements 3₁ to 3ₙ in accordance with the delay times 3₁ to 3ₙ. Consequently, the ultrasonic waves transmitted from the therapeutic transducer elements 3₁ to 3ₙ converge at the portion 7 to be treated, treating the lesion at the portion to be treated 7 by heating and cauterizing.

### Repetition of Treatment Operation: t4 to 5, t6 to f7, . . .

The above-described therapeutic operation is repeatedly performed at time intervals. At each repetition of this therapeutic operation, the cross-sectional image is re-imaged and the distance to the portion to be treated is re-measured for a definite period of time (Δt), the delay time τ₁ to τₙ of the driving pulses is calculated accordingly, and the focal point of the therapeutic transducer 3 is thus modified. In this manner, high-energy ultrasound can be transmitted from the therapeutic probe 1 while substantially confirming the state of cauterization in real time, whereby reliability and safety of the treatment is improved.

As treatment on one portion to be treated is completed, the operation returns to the beginning, where the therapeutic probe 1 is shifted so as to observe other portions to be treated, the focus is adjusted, and treatment is executed. In this manner, the treatment by ultrasonic transmission on a predetermined portion to be treated within the living body is completed. Incidentally, the time length of ultrasonic transmission from the therapeutic probe 3 is desirably set such that heat due to ultrasonic therapy is sufficiently diffused is and regions of the living body other than the portion to be treated are not damaged by heatapplied to the living body.

As described above, according to the embodiment shown in FIG. 1 and FIG. 3, the focal point of high-energy ultrasonic waves that are transmitted by the therapeutic transducer 3 can be varied, whereby it is unnecessary to prepare plural probes for various focal points and exchange them in performing treatment, and thus the time for the treatment can be shortened. In comparison with the conventional technique, the treatment on a lesion can be performed in a shorter time, whereby patient's pain can be reduced.

Further, as shown in FIG. 1, since the diagnostic probe 2 is provided in the middle of the therapeutic transducer 3, the portion to be treated is located on the cross-sectional image measured by the diagnostic probe 2, whereby treatment can be performed while constantly observing the portion to be treated within the living body. That is, it is desirable to integrally construct the diagnostic probe 2 and therapeutic transducer 3 so that the focal point of the ultrasonic beams transmitted by the therapeutic transducer 3 is located on the plane that the ultrasonic beams transmitted by the diagnostic probe 2 scan.

In the therapeutic transducer 3 according to the embodiment in FIG. 1, the ultrasonic transmission plane in the arranging direction of transducer elements is concavely formed and that in the width direction of transducer elements is flatly formed; however, the invention is not limited thereto. For example, the ultrasonic transmission plane in the width direction of transducer elements may also be concavely formed. Further, the whole area of the ultrasonic transmission plane may be flatly formed. The interior of the living body where the portion to be treated exists is observed by an ultrasonic tomography apparatus (not shown) that is connected with the diagnostic probe 2 applied to the body surface or to the surface of an organ when the viscera is opened up in an operation.

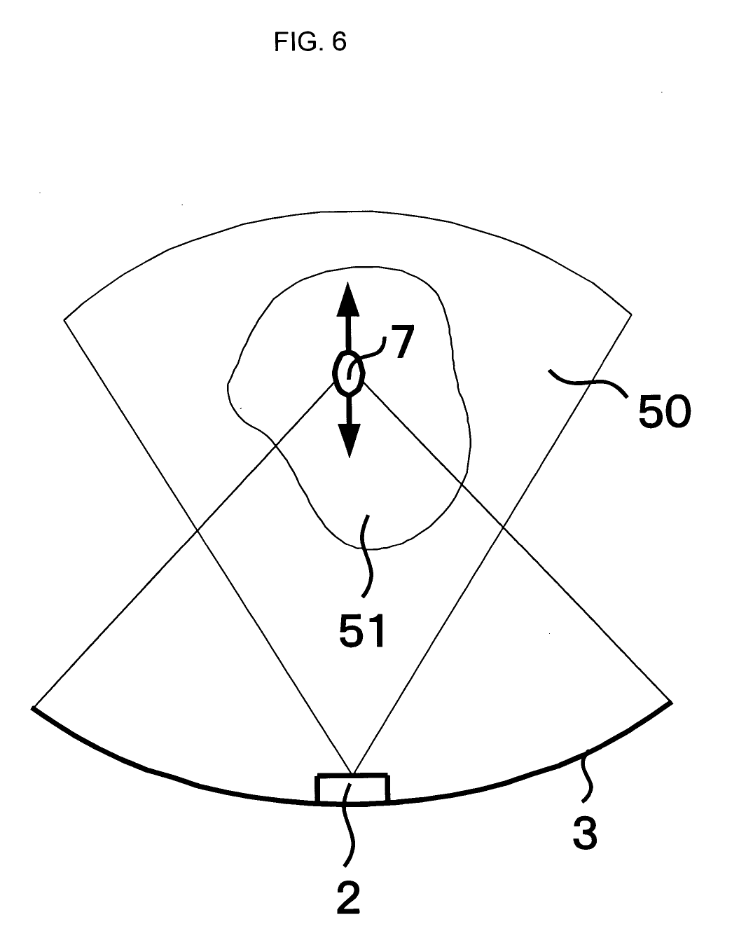

Next, when a lesion 51 appears on a cross-sectional image 50 of a living body obtained by the above ultrasonic tomography apparatus as shown in FIG. 6, signals supplied to the transducers 3₁ to 3ₙ are controlled by the variable focus control unit 6 such that the focal point of the therapeutic ultrasonic wave transducer 3, that is, the portion 7 to be treated, corresponds to the lesion 51, and a high-energy ultrasonic wave is transmitted to the above portion 7 to be treated.

In this regard, the diagnostic probe 2 and the therapeutic ultrasonic transducer 3 are constructed such that the portion 7 to be treated always shifts along the central portion of the cross-sectional image 50 in the depth direction.

The transmitted high-energy ultrasonic waves are focused in the area of the above portion 7 to be treated and converted into heat which cauterizes the lesion, thus performing treatment. Here, since the portion 7 to be treated within the living body is located on the cross-sectional plane scanned by the above diagnostic probe 2, the high-energy ultrasonic waves can be transmitted by the therapeutic ultrasonic transducer 3 while constantly observing the state of cauterizing in real time.

Further, as shown in FIG. 4, the cross-sectional image is taken at each repetition of therapeutic operation, whereby cauterization of a normal portion by mistake due to body movement or hand movement can be avoided, and safety can be thus improved.

According to the present invention, therapeutic ultrasonic waves can be transmitted to portions to be treated of various depths using one therapeutic probe. Further, ultrasonic therapy can be performed while substantially observing the portion to be treated through in a diagnostic image.

Scope of claims:

1. An ultrasonic therapeutic apparatus comprising a diagnostic probe; a therapeutic transducer; and a supporting member for supporting said diagnostic probe and said therapeutic transducer, said therapeutic transducer being divided into a plurality of transducer elements, and said plurality of transducer elements having an ultrasonic therapeutic probe that is connected with a distribution line to which the respective driving signals are supplied, this ultrasonic therapeutic apparatus further comprising a transmitting circuit for outputting ultrasonic-wave driving signals to the diagnostic probe of said ultrasonic therapeutic probe; a receiving circuit for receiving and processing a received signal which had been output by said diagnostic probe; an image processing unit for generating a diagnostic image based on the received signal processed by said receiving circuit; a display unit for displaying said diagnostic image generated by said image processing unit; a therapeutic wave transmitting circuit for outputting ultrasonic-wave driving signals to be supplied to said transducer elements of said therapeutic transducer of said ultrasonic probe; and a control unit for controlling said transmitting circuit, said receiving circuit, said image processing circuit, and said therapeutic wave transmitting circuit, wherein the focal point of ultrasonic beams transmitted by said therapeutic transducer is located on the plane which ultrasonic beams transmitted by said diagnostic probe scan.

2. An ultrasonic therapeutic apparatus according to claim 1, wherein the focal point of the ultrasonic beams transmitted by said therapeutic transducer of said ultrasonic therapeutic probe shifts in the depth direction of said diagnostic image.

3. An ultrasonic therapeutic apparatus according to claim 1, wherein the focal point of the ultrasonic beams transmitted by said therapeutic transducers is located in the central portion of said diagnostic image.

4. An ultrasonic therapeutic apparatus according to claim 1, wherein the focal point of the ultrasonic beams transmitted by said therapeutic probe shifts in a direction close to the depth direction.

5. An ultrasonic therapeutic apparatus according to claim 1, wherein said received signals are converted into a signal in which those received signals obtained from a desired portion within the living body are emphasized by adjusting the phase of the received signals.

6. An ultrasonic therapeutic apparatus according to claim 1, wherein a first diagnostic image obtained by ultrasonic imaging using said diagnostic probe is displayed, the portion to be treated is treated while observing the first diagnostic image, and a second diagnostic image is displayed.

7. An ultrasonic therapeutic apparatus according to claim 6, wherein, by using said control unit the distance to the portion to be treated is re-measured from the cross-sectional second diagnostic image, delay time of the driving pulses is calculated from the re-measurement, the focal point is adjusted, and the treatment is thus performed.

8. An ultrasonic therapeutic apparatus according to claim 7, wherein by using said control unit the ultrasonic imaging of the second diagnostic image and the ultrasonic therapy are sequentially performed.

9. An ultrasonic therapeutic apparatus according to claim 7 or 8, wherein, by using said control unit the ultrasonic waves are transmitted to the portion to be treated at time intervals.

10. An ultrasonic therapeutic apparatus according to claim 1, wherein said therapeutic transducer provided on the ultrasonic probe is divided in two and respectively arranged on either side with respect to the center of said diagnostic probe.

11. An ultrasonic therapeutic apparatus according to claim 1, wherein said therapeutic transducer of said ultrasonic therapeutic probe has a width direction and a longitudinal direction, and it is divided into a plurality of transducer elements in the longitudinal direction.

12. An ultrasonic therapeutic apparatus according to claim 11, wherein an ultrasonic transmission plane of said therapeutic probe is concavely formed in the longitudinal direction, and each line of the transducer elements is formed straight in the width direction.

13. An ultrasonic therapeutic apparatus according to claim 11, wherein the line of said transducer elements in the width direction is concavely formed.

14. An ultrasonic therapeutic apparatus according to claim 11, wherein said ultrasonic transmission plane is formed flatly or concavely.

15. An ultrasonic therapeutic apparatus according to claim 11, wherein the arranging direction of the transducer elements of said therapeutic transducer are perpendicular to the arranging direction of the transducer elements of said therapeutic probe.

16. An ultrasonic therapeutic apparatus comprising ultrasonic diagnostic imaging means; ultrasonic therapeutic means, and image display means for displaying received signals acquired from said ultrasonic diagnostic imaging means, wherein the focal point of ultrasonic beams transmitted by the ultrasonic therapeutic means is located on the plane scanned by ultrasonic beams transmitted by said diagnostic probe.

17. An ultrasonic therapeutic apparatus according to claim 16, wherein the focal point is located on the central portion of the diagnostic image.

18. An ultrasonic therapeutic apparatus according to claim 16, wherein the focal point shifts in a direction close to the depth direction of the diagnostic image.

19. An ultrasonic therapeutic apparatus according to claim 16, wherein the focal point shifts in the depth direction of the diagnostic image.

20. An ultrasonic therapeutic apparatus comprising a diagnostic probe; a therapeutic transducer, an ultrasonic probe which integrally has said diagnostic probe and said therapeutic transducer; a therapeutic pulse generating circuit; a therapeutic wave delay circuit; a diagnostic pulse generating circuit, a diagnostic wave transmission delay circuit; a transmission/reception separating circuit; a received wave phasing circuit; a signal processing unit; a digital scan converter; a monitor; a control unit for controlling said diagnostic probe, said therapeutic transducer, said therapeutic pulse generating circuit, said therapeutic wave delay circuit, said diagnostic pulse generating circuit, said diagnostic wave transmission delay circuit, said transmission/reception separating circuit, said received wave phasing circuit, said signal processing unit, and said digital scan converter; a console for giving a command to said control unit; and a signal amplifier.

21. An ultrasonic probe comprising a diagnostic probe; a therapeutic probe; an ultrasonic probe which integrally has said diagnostic probe and said therapeutic transducer; and a variable focus control unit for controlling ultrasonic waves of said diagnostic probe and said therapeutic transducer.
